(19) United States
(12) Patent Application Publication
(10) Pub. No.: US 2025/0108237 A1
(43) Pub. Date: Apr. 3, 2025

(54) SYSTEM AND METHOD FOR REDUCING NEURONAL DAMAGE IN STROKE AND HEART ATTACKS WITH TFUS

(71) Applicant: Sanmai Technologies, PBC, Sunnyvale, CA (US)

(72) Inventors: Tomokazu SATO, Minneapolis, MN (US); Christopher DAFT, Tucson, AZ (US); Timothy MULLEN, Hidden Meadows, CA (US)

(21) Appl. No.: 18/890,482

(22) Filed: Sep. 19, 2024

Related U.S. Application Data
(60) Provisional application No. 63/586,735, filed on Sep. 29, 2023.

(51) Int. Cl. A61N 7/02; A61N 7/00

(52) U.S. Cl. CPC A61N 7/02; A61N 2007/0021; A61N 2007/0052; A61N 2007/0078

(57) ABSTRACT

A transcranial focused ultrasound (tFUS) system treats and reduces brain damage in stroke, heart attack, and cardiac arrest patients. More generally, this tFUS system can also be used to treat neurotrauma where a physical issue can cause unhealthy neural activity changes (dysregulation), in particular over-excitation or excessive depolarization, that then leads to large-scale neural death or connectivity changes, potentially through a positive feedback loop in dysregulation. Thus, applications could include neurotrauma, including traumatic brain injury and concussions. The system allows for monitoring the efficacy of the treatment and adjusting the sonication parameters. The system integrates other diagnostic and treatment systems, such as MRI, fNIRS, hypothermic chamber, etc. A method utilizing a tFUS system treats and reduces brain damage in those suffering from stroke, heart attack, cardiac arrest, or neurotrauma.

### CROSS-REFERENCE TO RELATED APPLICATIONS

This application claims priority benefit of the U.S. Provisional Patent Application entitled “SYSTEM AND METHOD FOR REDUCING NEURONAL DAMAGE IN STROKE AND HEART ATTACKS WITH TFUS,” filed on Sep. 29, 2023, and having Ser. No. 63/586,735. The subject matter of this related application is hereby incorporated herein by reference.

### TECHNICAL FIELD

This invention relates to transcranial focused ultrasound systems (tFUS), particularly for treating and reducing brain damage caused by overexcitation or excessive depolarization of neurons, such as in stroke, heart attack, and neurotrauma patients.

### BACKGROUND

A stroke is a sudden loss of brain function caused by a disruption of blood flow to the brain, causing brain damage. This can happen when a blood vessel in the brain ruptures (hemorrhagic stroke) or a blood clot blocks a blood vessel (ischemic stroke). A stroke can cause lasting brain damage, long-term disability, or death. A stroke requires immediate treatment. The sooner treatment is started, the better the chances of recovery.

A heart attack (or myocardial infarction) happens when a part of the heart muscle doesn't get enough blood. Similarly, cardiac arrest is when a heart stops beating correctly. These conditions affect the heart's rhythm and ability to pump blood to the rest of the body. Heart attacks and cardiac arrest, like strokes, are medical emergencies and need immediate treatment. The heart will not support adequate blood flow; the brain must do without oxygen-rich blood. This damages the affected brain areas, causing a heart attack-acquired brain injury (ABI). First-aid treatment for heart attacks is relatively better established. Ordinary people can provide first aid treatment, e.g., CPR, or use an AED (automated external defibrillator) while waiting for medical personnel. A part of CPR's benefit is in the artificial circulation of blood and increased oxygenation of blood. However, auxiliary methods to support the health of brain cells could still provide great benefit.

Depending on the damage mechanism, concussions and traumatic brain injury (TBI) are thought to lead to long-lasting changes via similar mechanisms. Even if a brain cell is not directly damaged, damage to supporting structures such as blood vessels leading to the brain area or changes in brain activity nearby can lead to secondary damage.

However, first aid and treatment for stroke are complicated as the symptoms are ambiguous. Even if it is established that a patient is having a stroke (e.g., using F.A.S.T. (Facial weakness, Arm weakness, Speech problems, Time to call 911)), determining the location, stroke type, and treatment options is complicated.

These complexities of determining the stroke location and type mean that patients typically have to be diagnosed, carted to an open CT (computerized tomography) or MRI (Magnetic resonance imaging) system if available, have qualified surgeons arrive, and start treatment. When every minute is critical, starting the treatment would take, in the best case, 30 minutes or, at worst, multiple hours. In strokes, heart attacks, and some cases of TBI and concussions, blood flow disruption to the brain causes oxygen deprivation, causing local dysregulation of brain activity. In TBI and concussions, this may be the result of more direct physical damage. The resulting dysregulation of neural activity is a key mechanism by which damage can be induced more widely and long after initial trauma via positive feedback loops in activity dysregulation. There is a need for treatment options to decrease or help regulate dysfunctional brain activity. The treatment should start as quickly as possible to reduce brain damage, ideally before or while the patient is transported to a medical facility.

### BRIEF DESCRIPTION OF THE DRAWINGS

FIG. 1 is an example of a tFUS system 100 to treat stroke, heart attack, and neurotrauma patients.

FIG. 2 is an example block diagram of system 200 used in system 100, shown in FIG. 1.

FIG. 3 is an example flowchart showing a process performed using System 200 to treat stroke, heart attack, and neurotrauma patients.

FIG. 4 is an exemplary block diagram of tFUS system 210 used in System 200, shown in FIG. 2

FIG. 5 is an example inhibitory waveform generated by tFUS system 210.

### DETAILED DESCRIPTION

A transcranial focused ultrasound (tFUS) system treats and reduces brain damage in stroke, heart attack, and cardiac arrest patients. More generally, this tFUS system can also be used to treat neurotrauma where a physical issue can cause unhealthy neural activity changes (dysregulation), in particular over-excitation or excessive depolarization, that then leads to large-scale neural death or connectivity changes, potentially through a positive feedback loop in dysregulation. Thus, applications could include neurotrauma, including traumatic brain injury and concussions. The system allows for monitoring the efficacy of the treatment and adjusting the sonication parameters. The system integrates other diagnostic and treatment systems, such as MRI, fNIRS, hypothermic chamber, etc. A method utilizing a tFUS system treats and reduces brain damage in those suffering from stroke, heart attack, cardiac arrest, or neurotrauma.

Blood flow disruption to the brain causes brain damage in strokes, heart attacks, and some cases of TBI and concussions. A key mechanism is often the dysregulation of neural activity. This could result from the blood flow disruption or from more direct mechanisms of physical damage. This causes a positive feedback loop with increased metabolism and neural activity as follows:
Blood flow disruption or more direct physical damage mechanisms disrupt a neuron's voltage across its cell membrane. Blood flow disruption may occur via altering cell metabolism or affecting access to energy required for membrane potential maintenance. Direct mechanical damage may include mechanical stretching of neurons and neural processes, leading to direct depolarization.
The cells lose maintenance of their hyperpolarized (negative voltage inside the neurons relative to extracellular space) state: I.e., they get depolarized.
Depolarization leads to the opening of voltage-gated sodium channels, leading to further depolarization.
Depolarization leads to the opening of voltage-gated calcium channels.
This leads to neurotransmitter release; most cells are excitatory, releasing excitatory neurotransmitters that lead to downstream neural activity.
This further increases local neural activity, which compounds on top of the metabolic disruption caused by alterations in blood flow.
This positive feedback loop of uncontrolled increased neural activity (and increased metabolic waste) leads to excitotoxicity.

Other related mechanisms, including increases in extracellular or interstitial K+ ion concentrations, calcium-independent glutamate release, etc., that in general can contribute to the depolarization of cell membranes and can contribute in a feedback mechanism are also implicated in playing a role in this positive feedback loop of cellular depolarization. These mechanisms would also be candidate targets for therapeutic tFUS.

These intense depolarizations may spread from the foci of damage in stroke or TBI cases and be seen as a spreading wave of activity changes reaching far beyond the original foci. This wave of self-propagating cellular depolarization is often called spreading depolarization or spreading depression. It is thought to be one mechanism by which brain-wide changes may occur in some instances. Such intensive depression can lead to changes in metabolic demands at the single neuron level that can trigger a host of downstream signaling, including apoptosis; metabolic dysfunction at a more significant population level; and changes in neurovascular function in response to these changes. Supporting cells in the brain, such as glia, may also be affected or overwhelmed in their capacity to perform normal support and upkeep functions.

To simplify wording moving forward, when stroke, heart attack, or neurotrauma are mentioned, we refer to the broader set that includes other diseases and injuries causing brain injury using the same or similar key mechanism of excessive depolarization of cells or overexcitation of cells, including TBI, concussion, and cardiac arrest.

Transcranial focused ultrasound (tFUS) is a neuromodulation technology in which low-intensity ultrasound is non-invasively transmitted into shallow or deep brain regions. TFUS-based inhibitory waveforms are typically continuous, high-frequency, or different from a brain area's natural rhythm. tFUS can be used to decrease or help regulate dysfunctional brain activity. In an embodiment of the invention, one or more waveforms are selected from a library of tFUS-based waveforms (inhibitory waveforms) used to regulate or inhibit dysfunctional brain activity (also referred to as neuromodulation). Inhibitory waveforms tend to have extended duration of individual pulses. Typically, pulses will be at least on the order of 100 us and often up to 1 s or continuous. The inhibitory waveforms can vary in sonication parameters such as tone burst duration (TBD), pulse repetition frequency (PRF), acoustic intensity, acoustic frequency, duration (NTB or number of tone bursts), etc. Feedback on the efficacy of the inhibitory waveform is obtained by monitoring the neural activity, and the waveform is adjusted. A tFUS system can perform US-based (ultrasound-based) imaging (neural activity measure) with skull aberration correction in an embodiment. In a different embodiment, the neural activity is measured using EEG (electroencephalogram), fNIRS (Functional Near-Infrared Spectroscopy), or fMRI (Functional magnetic resonance imaging). Inhibitory waveforms work due to multiple mechanisms, such as:
Small depolarizations, due to the inactivation of sodium channels, are known to disrupt firing. The refractory period after a neuron fires an action potential relies on this mechanism.
Opening of K+ and Cl− channels also leads to hyperpolarization and clamping to negative voltages under normal ionic circumstances.
Activating inhibitory cells to release inhibitory neurotransmitters (GABA, glycine) can reduce local neural activity.
Off-target activation or inhibition (i.e., modulation of a brain region that feeds into the area with stroke is suppressed or activated depending on the connection).

In our discussion, we will broaden the term of inhibitory waveforms to those that inhibit neural firing, inhibit the self-sustained spread of depolarization, or inhibit depolarization at the local and single-cell level, as depending on the mechanism, severity, and timepoint of the brain injury, and even specific timepoint and spatial location relative to the injury foci or a spreading wave of neural activity change, the effect we wish to inhibit may differ slightly. In addition, as local ionic concentrations may undergo drastic changes in some circumstances at specific locations and time points, activation of K+ ion channels, for example, may have an excitatory effect instead of a normal inhibitory effect. These considerations are taken broadly when describing inhibitory waveforms, where inhibition implies inhibiting the dysfunction caused by underlying issues and causing subsequent neural damage or dysfunction.

In one embodiment, the same ultrasound system used for tFUS or US-based imaging is also used for ultrasound-mediated cautery of bleeds, the disintegration of ischemic clots, and delivery of drugs via BBB-disruption (blood-brain barrier) or nanoemulsions. A completely orthogonal mechanism of reducing metabolic activity, hypothermia of the body, head, or portions of the head using hypothermic chambers or the local application of cooling patches to the carotid artery or areas of stroke or trauma may also be used. As there are many mechanisms to inhibit dysfunctional brain activity, the treatments need not be exclusive; multiple treatments can be tried simultaneously, one after the other, while monitoring neural activity for feedback on efficacy.

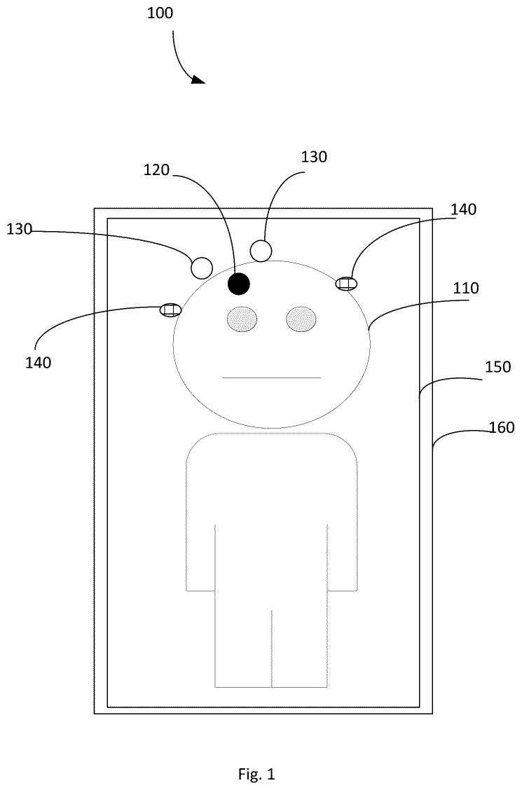

FIG. 1 is an example of a tFUS system 100 to treat stroke or heart attack patients. tFUS system 100 is capable of skull aberration correction and can be used for US imaging and US neuromodulation. tFUS system 100 can be used for direct ultrasound-based neuromodulation, including inhibition of neural activity. In addition, tFUS system 100 can be used for other orthogonal, ultrasound-based therapeutic modalities, including ischemic clot-busting, HIFU cautery of bleeds (high-intensity focused ultrasound), or drug (inhibitory neurotransmitters, anti-oxidants, etc.) delivery by BBB-disruption or nanoemulsions. In an embodiment, System 100 is portable and can be transported to a patient's location via an ambulance or other specialized vehicle. This helps to diagnose, start treatment, and reduce brain damage immediately. Referring to FIG. 1, patient 110 has a stroke at location 120. One or more ultrasound transducers or patches of transducer arrays 130 are attached to the patient's head. Transducers 130 can be used for US imaging and neuromodulation. Optional EEG electrodes 140 are attached to the patient's head to provide feedback on the efficacy of the tFUS treatment or to aid with localization. In an embodiment, fNIRS is used instead of EEG to provide feedback on the efficacy of the tFUS treatment or to aid with localization. System 100 includes an optional imaging system 150, based on imaging modalities other than ultrasound, to confirm diagnosis, aid with the localization of the stroke, or provide feedback as to the efficacy of treatment. Imaging system 150 can be an MRI, RF, microwave, or eddy current imaging system. Patient 110 can be enclosed in an optional hypothermic chamber 160. Optional Peltier cooling (not shown in the figure) or patches of circulating cold fluid for local head cooling can also be deployed. The cooling patches are deployed near infarct, bleeding, or damage, especially if superficial or on the major arteries leading to the brain. The cooling may be applied on the skin or invasively. System 100 can include an IV (intravenous) system (not shown in the figure) to deliver medications, etc.

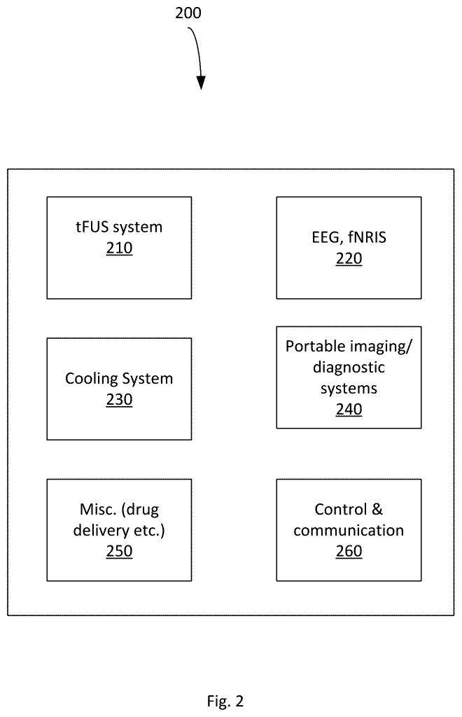

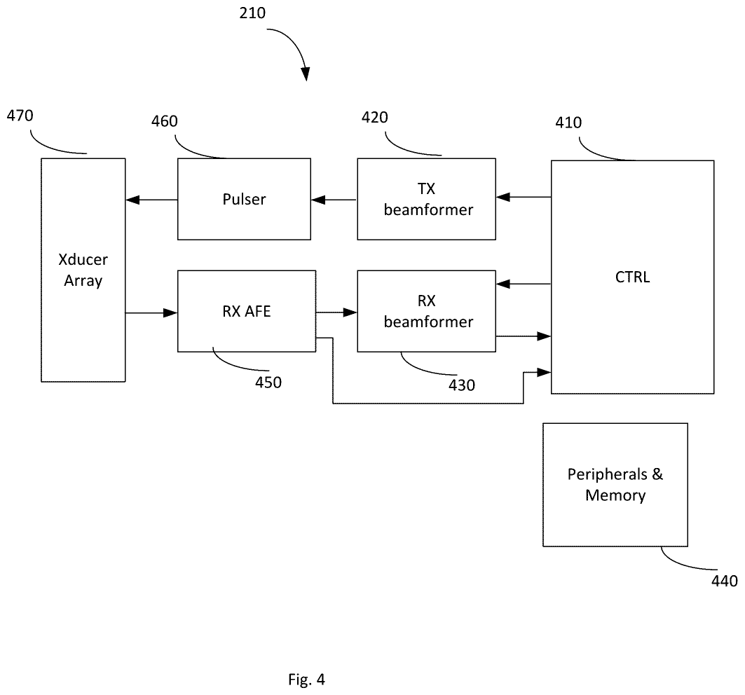

FIG. 2 is an example block diagram of system 200 used in system 100, shown in FIG. 1. Referring to FIG. 2, System 200 includes a tFUS system 210 used for US imaging and neuromodulation. tFUS system 210 includes one or more transducers attached to the patient's skull or head. A human skull is of varying thickness and density and causes non-uniform refraction, attenuation, and aberration to the incident and reflected ultrasound waves. tFUS system 210 can make corrections caused by the varying skull thickness and density. More details on the tFUS system 210 are shown in FIG. 4. System 200 includes EEG or fNIRS system 220 to provide feedback on the efficacy of the tFUS treatment or to aid with localization. Imaging and diagnostic systems 240, such as an MRI, RF, microwave, or eddy current sensing system, can also be used to diagnose and locate the stroke and provide feedback on treatment efficacy, in addition to ultrasound-based means. Cooling system 230 provides cooling that can be used for hypothermic chamber 160 or cooling patches. A miscellaneous block 250 is used for drug delivery (IV system), oxygen delivery, etc. A control & communication system 260 is used for the overall control of System 200. It includes necessary hardware such as CPUs, memory, storage, and power supplies to maintain, control, and debug system 200. Control and Communication System 260 includes necessary UI (user interface) interfaces and peripherals such as monitors, keyboards, mice, etc., to display scan images (US, MRI, EEG, fNIRS, etc.) and control the various systems (tFUS 210, EEG 220, imaging/diagnostic 240, etc.). System 200 is portable and can be transported to a patient's location via an ambulance or other specialized vehicle. This helps to diagnose, start treatment, and reduce brain damage immediately. System 200 records the location and type of stroke, various treatments performed, and treatment efficacy for future use (e.g., supervised learning). In an embodiment, Control & Communication System 260 stores a library of tFUS-based inhibitory waveforms used to regulate or inhibit dysfunctional brain activity. In an embodiment, System 200 suggests the inhibitory waveforms and skull aberration correction parameters to be used (for example, using supervised learning and real-time feedback from EEG, fNIRS, MRI, or other methods that can measure changes in neural activity, metabolism, blood flow, or other related signatures of treatment efficacy).

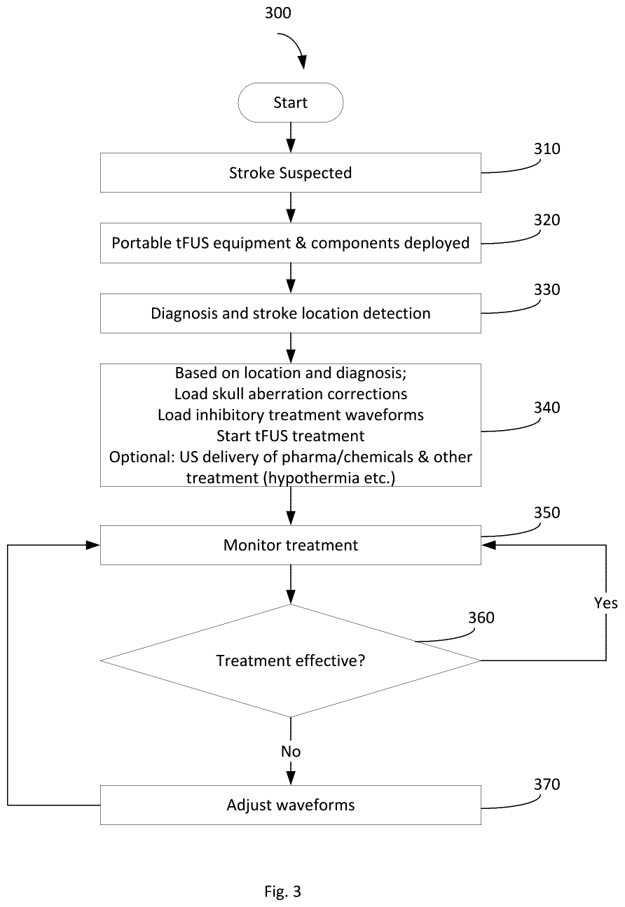

FIG. 3 is an example flowchart 300 showing a process performed using System 200 to treat stroke, heart attack, cardiac arrest, or neurotrauma patients. Referring to FIG. 3, in operation 310, a patient is suspected to be suffering from the conditions mentioned earlier, and emergency services such as 911 are contacted by the patient or on behalf of the patient. In operation 320, tFUS equipment and other equipment (e.g., System 200) are deployed.

In operation 330, if stroke is suspected, diagnosis and stroke location detection are performed. In an embodiment, US-based imaging (using tFUS system 210) is used for diagnosis and detecting stroke location. This is done using US imaging of blood flow, tissue characterization, etc. In another embodiment, the diagnosis and stroke location diagnosis are performed by other equipment such as imaging/diagnostic systems 240 or EEG, fNIRS 220. In a different embodiment, diagnosis and detection are done using both tFUS system 210 and other diagnostic systems 240 and EEG, fNIRS 220. The type of stroke (ischemic or hemorrhagic) is also diagnosed during operation 330. It is important to note that while blood flow disruption may not be a primary mechanism of brain injury with the other conditions mentioned, imaging and diagnostic capabilities can be critical. These would include deeper brain hemorrhaging secondary to TBI; cases where accurate assessment of whether stroke or cardiac issues are involved due to vague symptoms or loss of consciousness by the patient; or simultaneous cardio-cerebral infarction, thought to be rare before the coronavirus pandemic but increasingly recorded in medical literature. Thus, while Operation 330 may not always be necessary, it may be prudent to perform in some instances.

In operation 340, the location and diagnosis information gathered from operation 330 are used to determine the treatment steps. Based on stroke type and location, various treatment options are considered. For example, in the case of ischemic strokes, the treatment plan includes the US-based dissolving of the clot, whereas in the case of hemorrhagic strokes, treatment includes US-based cautery of the bleeding. Other treatments include US delivery of drugs via BBB-disruption (blood-brain barrier) or nanoemulsions, therapeutic hypothermia of the patient's head or whole body, etc. A human skull is of varying thickness and density and causes non-uniform refraction, attenuation, and aberration to the incident and reflected ultrasound waves. Based on the stroke's location and the transducers' placement, skull aberration correction parameters are loaded from memory. The skull aberration correction parameters correct the tFUS neuromodulation and imaging waveforms for the varying skull thickness and density. Inhibitory waveforms for treatment are loaded from memory and the treatment is started. In an embodiment, System 200 suggests the inhibitory waveforms and skull aberration correction parameters to be used (for example, using supervised learning).

While strokes are local, the dysregulation can spread through the aforementioned spreading depolarization or spreading depression mechanisms. In addition, brain regions upstream or downstream of the areas most directly affected by the stroke would also be targets of interest. Thus, even though strokes cause problems through a localized mechanism initially, the entirety of the brain can be considered targets for most efficacious treatment. In the case of a heart attack or cardiac arrest, the whole brain does not receive adequate oxygenated blood. The affected areas of the brain begin to die, some possibly earlier than others, so the treatment for more critically affected brain areas is similar to one used for strokes. In the case of TBI, traumatic brain injury (TBI) can cause swelling (edema) in the brain, can increase intracranial hypertension (ICP), and can worsen the injury. Cell death can occur minutes to hours after the injury, and the harmful effects can last for 72 hours or longer. Similar inhibitory waveforms can be used to decrease the effects of swelling, ICP, etc. Similar to the treatment of stroke, it is likely there will be affected areas near the injury site that require more treatment than others, but due to the spreading damage mechanisms that are possible, all parts of the brain should be considered targets for treatment.

During Operation 340, even if the location of the issue is known, such as in TBI, or suspected to be general, such as in heart attacks, skull aberration corrections and other steps must be taken even if there is no single specific location or diagnosis relevant for targeting. In addition, as mentioned before, spreading depolarization/depression can be detected via EEG or fNIRS, and said information can be used for locating treatment and different waveforms. The waveforms used may differ depending on whether the target is a location of current depolarization, likely upcoming depolarization, or has just recently experienced depolarization and has somewhat recovered.

In addition, even for generalized brain injury mechanisms (heart attack, concussion, etc.), skull aberration and the ability to localize and move the target of the therapeutic tFUS waveform are useful. This way, the tFUS waveform may be aimed at multiple regions spanning parts or the whole brain. It may be done in an ordered (raster scanning, spiral scanning), pseudorandom, random, or feedback-mediated manner. This latter approach utilizes information gained from ultrasound imaging, EEG, fNIRS, or other diagnostic imaging techniques that may highlight regions of the brain most in need of tFUS. This could include the rapid detection of the start of a spreading depolarization and the suppression or disruption of its spread.

In operation 350, the efficacy of the treatment is monitored using tFUS imaging or other imaging systems such as EEG, fNIRS, MRI, etc. In addition, these techniques may be used to track their progression in moving changes in cellular depolarization.

In operation 360, it is determined whether the treatment is effective. If the treatment is ineffective, the following operation is operation 370, where the inhibitory waveforms and treatment options are adjusted. For example, if the EEG indicates a high amount of activity from the target brain region, one can adjust timing, intensity, or spatial/targeting parameters of the ultrasound waveform and field shape. These could include increased intensity, adjusting the pulse repetition frequency, or rastering the ultrasound beam around to see if a better target location can be found. If the treatments are effective, flowchart 300 reverts to operation 350 to continuously monitor the treatment.

In an embodiment of the invention, multiple inhibitory waveforms are used simultaneously. In this case, one inhibitory waveform after the other is used while monitoring neural activity for feedback on efficacy. When the treatment uses multiple inhibitory waveforms, in operation 340, the first waveform is loaded, the treatment starts, and then the efficacy is monitored in operation 350. The flowchart reverts to Operation 340 for loading the second waveform, etc.

FIG. 4 is an exemplary block diagram of tFUS system 210 used in System 200, shown in FIG. 2. tFUS system 210 is used for ultrasound-based imaging and generating tFUS-based inhibitory waveforms to regulate or inhibit dysfunctional brain activity. For ultrasound-based imaging, tFUS system 210 must have components to generate and receive ultrasound signals. Referring to FIG. 4, System 210 consists of an array of transducers 470 (Xducer Array). Transducers convert electrical signals to mechanical signals and vice versa. Xducer Array 470 includes ultrasound transmitters and receivers. Array 470 converts electrical signals from Pulser 460 to ultrasound signals to be transmitted into the brain. The reflected ultrasound signal generated by the brain and skull is received by array 470, converted into an electrical signal and sent to RX AFE 450. Array 470 is placed on the skull (as shown in FIG. 1) and can be placed in one or more locations on the skull to image or neuromodulate spatially diverse brain locations. Array 470 can be configured to consist of various numbers of transmitters and receivers; individual elements may be used as transmitters only, receivers only, or for both operations. Individual elements within the Array 470 can be configured to transmit ultrasound signals for imaging and neuromodulation (or inhibitory waveforms). Imaging waveforms are typically short, usually 1 or a few cycles. Inhibitory or neuromodulation waveforms can be long tone bursts. TX beamformer 420 and Pulser 460 control the transmitted ultrasound signals direction, depth, pulse pattern, duration, amplitude, and delays. Based on the array 470′s location on the skull, CTRL (control) 410 generates this information; this information is used to correct any skull aberrations and generate neuromodulation waveforms. Similarly, during the ultrasound reception part used for imaging, CTRL 410 provides the aberration correction computation information, the beam formation delays, and other processing information (such as impulse response filter information) to RX AFE 450 and RX beamformer 430.

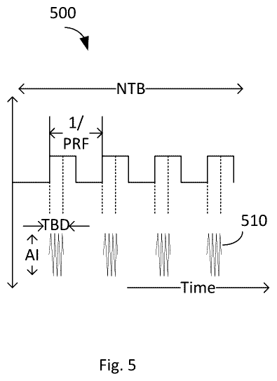

FIG. 5 is an example inhibitory waveform generated by tFUS system 210. Referring to FIG. 5, tFUS system 210 can generate a repeating pulse or tone (marked as 510 in the figure). The acoustic frequency (AF, not marked in the figure) and amplitude (AI—acoustic intensity), TBD (tone burst duration or the duration of the pulse) can be modified. The pulse 510 is repeated with a period of 1/PRF (pulse repetition frequency). The total duration or NTB (number of tone bursts) can also be controlled. tFUS system 210 has a library of inhibitory waveforms where various parameters, such as AF, AI, TBD, PRF, NTB, etc., are fixed and based on commonly used inhibitory waveforms. Operators can select an appropriate waveform based on the diagnosis. In a different embodiment, operators can adjust the parameters (AF, AI, TBD, PRB, NTB, etc.) in real-time and evaluate the efficacy. This can be done during Operation 370 of flowchart 300, shown in FIG. 5. In a different embodiment, tFUS system 210 can suggest an inhibitory waveform based on the diagnosis.

Inhibitory and imaging waveforms and skull aberration correction parameters are stored in Peripherals & Memory 440 and can be loaded by CTRL 410. Peripherals include wired and wireless interfaces to update the information stored in System 210 or to transmit data to other external systems (such as a server). Storage stores programs, instructions, and data needed for CTRL 410. CTRL 410 controls the overall operation of system 210. It may include one or more FPGAs (Field Programmable Gate Arrays), GPUs (Graphics Processing Units), DSPs (Digital Signal Processors), CPUs (Central Processing Units), Neural Network processors, or AI processors.

What is claimed is:

1. A system comprising:
at least one ultrasonic transducer array; and
at least one processor coupled to the at least one ultrasonic transducer array, wherein the at least one processor generates an inhibitory waveform based upon an acoustic frequency (AF), acoustic intensity (AI), or tone burst duration (TBD) specified by a library of inhibitory waveforms accessible to the at least one processor and wherein the at least one ultrasonic transducer array transmits the inhibitory waveform.

2. The system of claim 1, wherein the inhibitory waveform is modified based upon a plurality of skull aberration correction parameters.

3. The system of claim 1, wherein the plurality of skull aberration correction parameters are calculated by the at least one processor based upon ultrasound reflections received by the at lest one ultrasonic transducer array.

4. The system of claim 1, further comprising a pulser coupled to the processor, the pulser configured to transmit electrical signals corresponding to the inhibitory waveform to the at least one ultrasonic transducer array, wherein the at least one ultrasonic transducer array converts the electrical signals to ultrasonic signals.

5. The system of claim 1, further comprising at least one electroencephalogram (EEG) electrode configured to detect electrical activity in response to the inhibitory waveform and transmit the electrical activity to the at least one processor.

6. The system of claim 1, further comprising a Functional Near-Infrared Spectroscopy (fNIRS) probe configured to detect brain dynamics in response to the inhibitory waveform and transmit the brain dynamics to the at least one processor.

7. The system of claim 1, further comprising a cooling system.

8. The system of claim 7, wherein the cooling system comprises a hypothermic chamber or at least one cooling patch.

9. The system of claim 1, further comprising an intravenous drug delivery system or an oxygen delivery system.

10. The system of claim 1, wherein the at least one processor adjusts the inhibitory waveform in response to feedback obtained in response to the inhibitory waveform.

11. The system of claim 10, wherein the at least one processor adjusts the inhibitory waveform by modifying at least one of the acoustic frequency (AF), acoustic intensity (AI), or tone burst duration (TBD).

12. A method comprising:
generating an inhibitory waveform based on a library of inhibitory waveforms and a patient;
transmitting the inhibitory waveform to at least one ultrasound transducer;
obtaining a response to the inhibitory waveform; and
adjusting the inhibitory waveform based upon the response.

13. The method of claim 12, further comprising cooling a patient associated with the inhibitory waveform using a cooling system.

14. The method of claim 12, further comprising determining a location to apply the at least one ultrasound transducer based upon imaging of at least one of blood flow or tissue characterization.

15. The method of claim 14, wherein the location corresponds to a stroke occurring in a patient.

16. The method of claim 12, wherein the inhibitory waveform for treatment, where the inhibitory waveform is generated based upon an acoustic frequency (AF), acoustic intensity (AI), or tone burst duration (TBD) specified by the library of inhibitory waveforms.

17. The method of claim 12, further comprising modifying the inhibitory waveform based upon skull aberration parameters.

18. The method of claim 12, further comprising monitoring an efficacy of the inhibitory waveform based upon electrical activity in response to the inhibitory waveform obtained using at least one electroencephalogram (EEG).

19. The method of claim 12, further comprising monitoring an efficacy of the inhibitory waveform based upon a Functional Near-Infrared Spectroscopy (fNIRS) probe configured to detect brain dynamics in response to the inhibitory waveform.

20. The method of claim 12, further comprising monitoring an efficacy of the inhibitory waveform based on metabolic imaging, blood flow imaging or changes in neurotransmitter concentrations.

21. The method of claim 12, further comprising causing delivery of an intravenous drug with the inhibitory waveform.
